(19) United States
(12) Patent Application Publication
(10) Pub. No.: US 2026/0132441 A1
(43) Pub. Date: May 14, 2026

(54) METHOD FOR CONCENTRATING PATHOGENS OR NUCLEIC ACID USING ACID ACTIVATED BENTONITE

(71) Applicant: INDUSTRY-ACADEMIC COOPERATION FOUNDATION, YONSEI UNIVERSITY, Seoul (KR)

(72) Inventors: Yong SHIN, Seoul (KR); Eun Yeong LEE, Guri-si (KR)

(21) Appl. No.: 19/387,022

(22) Filed: Nov. 12, 2025

(30) Foreign Application Priority Data
Nov. 14, 2024 (KR) 10-2024-0162152
Oct. 16, 2025 (KR) 10-2025-0149288

(51) Int. Cl. C12Q 1/6806; C01B 33/40; C12Q 1/02; C12Q 1/70

(52) U.S. Cl. CPC C12Q 1/6806; C01B 33/40; C12Q 1/02; C12Q 1/70; C01P 2006/12

(57) ABSTRACT

An embodiment relates to a composition for concentrating nucleic acid or pathogen, a kit for concentration, a method for concentration, and a method for preparing acid-activated bentonite for concentration, the composition comprising acid- and silane compound-modified bentonite and homobifunctional imidoesters as active ingredients. According to the embodiment, it was confirmed that the bentonite adsorbs pathogens and nucleic acids stably and can rapidly elute highly purified nucleic acids. Therefore, the composition can be immediately applied to subsequent analyses such as real-time PCR (qPCR), and can rapidly process large-volume samples using only simple tools, thereby providing both cost efficiency and accessibility. Accordingly, the embodiment can be effectively applied to various analytical methods utilizing nucleic acids and pathogens.

### CROSS REFERENCE TO RELATED APPLICATION

This application claims the priority of Korean Patent Application No. 10-2024-0162152 filed on November 14, 2024, and Korean Patent Application No. 10-2025-0149288 filed on October 16, 2025, in the Korean Intellectual Property Office, the disclosure of which is incorporated herein by reference.

### BACKGROUND OF THE INVENTION

### FIELD OF THE INVENTION

The present invention relates to a composition for concentrating nucleic acid or pathogen comprising acid- and silane compound-modified bentonite and homobifunctional imidoesters as active ingredients, a kit for concentration, a method for concentration, and a method for preparing acid-activated bentonite for concentration.

### DESCRIPTION OF THE RELATED ART

High-quality nucleic acid purification is an essential step in various fields of research such as medicine, microbiology, and immunology. Efficient separation of nucleic acids is indispensable for accurate subsequent applications, including diagnosis, genetic, and therapeutic studies. Silica-based solid-phase extraction (SPE) methods have been widely used due to their extraction efficiency and ease of use. However, they have drawbacks such as inhibitory effects of chaotropic ions in downstream analysis and a reduced recovery rate of nucleic acids caused by the limited surface area. In addition, conventional nucleic acid extraction processes require multiple manipulation steps and large instruments, making the procedure labor-intensive and demanding skilled operators.

To solve these problems, research has focused on the development of advanced SPE materials with high surface area and improved binding efficiency, as well as the optimization of simplified and user-friendly extraction protocols. Although microfluidic systems and novel adsorbent materials have been applied to SPE technology to address low recovery yields, these approaches still suffer from challenges such as low reproducibility and dependency on specialized laboratory equipment. Moreover, although simplified and cost-efficient nucleic acid extraction technologies have been proposed for on-site diagnostics, they exhibit poor extraction efficiency for small quantities of biomarkers such as miRNA or viral RNA. Therefore, there is a need for the development of an SPE technology capable of effectively solving issues of reproducibility, equipment dependency, and nucleic acid recovery rate, thereby improving the reliability and accessibility of nucleic acid purification methods.

Relevant Non-Patent Literature: Archives of Biochemistry and Biophysics, Vol. 128, Issue 3, pp. 579–582.

### SUMMARY OF THE INVENTION

One aspect of the present invention is to provide a composition for concentrating nucleic acid or pathogen comprising acid- and silane compound-modified bentonite and homobifunctional imidoesters as active ingredients.

Another aspect of the present invention is to provide a kit for concentrating nucleic acid or pathogen comprising the above composition.

A further aspect of the present invention is to provide a method for concentrating nucleic acid or pathogen comprising adding and mixing the above composition with a biological sample, and separating nucleic acid or pathogen from the mixture.

Another aspect of the present invention is to provide a method for preparing bentonite for use in nucleic acid or pathogen concentration.

Another aspect of the present invention is to provide a modified bentonite comprising amino (-NH₂) or amine (-NHR) functional groups on its surface.

To achieve the above objectives, the present invention provides a composition for concentrating nucleic acid or pathogen comprising acid- and silane compound-modified bentonite and homobifunctional imidoesters as active ingredients.

In one embodiment of the present invention, the acid may be one or more selected from the group consisting of perchloric acid, hydroiodic acid, hydrobromic acid, hydrochloric acid, sulfuric acid, nitric acid, chloric acid, p-toluenesulfonic acid, methanesulfonic acid, fluoroantimonic acid, fluorosulfonic acid, and triflic acid.

In another embodiment, the silane compound may be one or more selected from the group consisting of (3-aminopropyl)triethoxysilane (APTES), (3-aminopropyl)trimethoxysilane, (1-aminomethyl)triethoxysilane, (2-aminoethyl)triethoxysilane, (4-aminobutyl)triethoxysilane, (5-aminopentyl)triethoxysilane, (6-aminohexyl)triethoxysilane, 3-aminopropyl(diethoxy)methylsilane (APDMS), N-[3-(trimethoxysilyl)propyl]ethylenediamine, N-[3-(trimethoxysilyl)propyl]diethylenetriamine, [3-(2-aminoethylamino)propyl]trimethoxysilane (AEAPTMS), and 3-[(trimethoxysilyl)propyl]diethylenetriamine (TMPTA).

In another embodiment, the homobifunctional imidoester may be one or more selected from the group consisting of dimethyl adipimidate (DMA), dimethyl pimelimidate (DMP), dimethyl suberimidate (DMS), and dimethyl 3,3'-dithiobispropionimidate (DTBP).

In another embodiment, the nucleic acid may be one or more selected from the group consisting of DNA, RNA, miRNA, mRNA, siRNA, tRNA, sgRNA, and shRNA.

In another embodiment, the pathogen may be a microorganism.

In another embodiment, the microorganism may be one or more selected from the group consisting of virus, bacterium, fungus, and protozoa.

The present invention also provides a kit for concentrating nucleic acid or pathogen comprising the above composition.

The present invention also provides a method for concentrating nucleic acid or pathogen comprising adding and mixing the above composition with a biological sample, and separating nucleic acid or pathogen from the mixture.

In one embodiment, the biological sample may be one or more selected from the group consisting of blood, serum, plasma, lymph fluid, cerebrospinal fluid, body fluid, saliva, feces, urine, cerebrospinal fluid, gastric secretion, ascites, nasal secretion, sputum, pharyngeal exudate, and tissue.

In another embodiment, the composition may be added at a concentration of 0.2–5 mg/ml per 1 mL of the biological sample.

In another embodiment, the mixing may comprise incubation for 15–60 minutes.

In another embodiment, the mixing may be performed at 45–60°C.

In another embodiment, the separating may comprise injecting a high-pH elution buffer to separate the nucleic acid from the bentonite.

In another embodiment, the high-pH elution buffer may have a pH of 10.5–12.

The present invention also provides a method for preparing bentonite for concentrating nucleic acid or pathogen, comprising: (i) treating bentonite with an acid to obtain activated bentonite; and (ii) modifying the activated bentonite with a silane compound.

In one embodiment, the acid may be one or more selected from the group consisting of perchloric acid, hydroiodic acid, hydrobromic acid, hydrochloric acid, sulfuric acid, nitric acid, chloric acid, p-toluenesulfonic acid, methanesulfonic acid, fluoroantimonic acid, fluorosulfonic acid, and triflic acid.

In another embodiment, the acid treatment in step (i) may be performed using an acid concentration of 0.5–10%.

In another embodiment, the silane compound may be one or more selected from the group consisting of (3-aminopropyl)triethoxysilane (APTES), (3-aminopropyl)trimethoxysilane, (1-aminomethyl)triethoxysilane, (2-aminoethyl)triethoxysilane, (4-aminobutyl)triethoxysilane, (5-aminopentyl)triethoxysilane, (6-aminohexyl)triethoxysilane, 3-aminopropyl(diethoxy)methylsilane (APDMS), N-[3-(trimethoxysilyl)propyl]ethylenediamine, N-[3-(trimethoxysilyl)propyl]diethylenetriamine, [3-(2-aminoethylamino)propyl]trimethoxysilane (AEAPTMS), and 3-[(trimethoxysilyl)propyl]diethylenetriamine (TMPTA).

The present invention also provides a modified bentonite comprising amino (-NH₂) or amine (-NHR) functional groups on the surface.

In another embodiment, the modified bentonite may have a specific surface area of 150 m²/g to 300 m²/g.

The inventors of the present invention developed an aminated solid-phase extraction and concentration method for pathogens or nucleic acids based on acid-activated bentonite and confirmed that the nucleic acid extraction efficiency was high. This method enables stable adsorption of pathogens and nucleic acids and rapid elution of highly purified nucleic acids. Accordingly, the extracted nucleic acids can be directly applied to subsequent analyses such as real-time PCR (qPCR). Furthermore, since the process can be carried out using only simple tools, such as a pipette and a centrifuge, large quantities of samples can be rapidly processed, thereby providing both cost efficiency and accessibility. Therefore, the method of the present invention can be applied to various analytical approaches utilizing nucleic acids or pathogens.

### BRIEF DESCRIPTION OF THE DRAWINGS

FIG. 1 illustrates a schematic overview of the procedure for pathogen and nucleic acid separation using a pH-dependent reversible crosslinking reaction employing an acid-activated sulfuric bentonite (ASAB) solid phase modified with APDMS. (a) of FIG. 1 shows the process of modifying raw bentonite into ASAB. (b) of FIG. 1 shows that when homobifunctional imidoesters (HI) are used as crosslinkers, the primary amine-derived polarity exposed on the ASAB surface can form strong covalent bonds with pathogens or nucleic acids. (c) of FIG. 1 shows a schematic workflow of the pathogen concentration and nucleic acid extraction process using ASAB through a pH-dependent reversible crosslinking reaction.

FIG. 2 shows the optimization results of the ASAB-based SPE system using HI group reagents under various conditions for nucleic acid extraction. (a) of FIG. 2 shows the CT values obtained when using various clay minerals as solid-phase matrices (DE: diatomite, Zeo: zeolite, Bento: bentonite, Hallo: halloysite, Montmo: montmorillonite, Kao: kaolinite) for DNA extraction. (b) of FIG. 2 shows CT values of DNA extracted using sulfuric acid-activated bentonites of three different concentrations (Bento: raw bentonite, 1: 1% H₂SO₄-activated bentonite, 5: 5% H₂SO₄-activated bentonite, 10: 10% H₂SO₄-activated bentonite). (c) of FIG. 2 shows CT values obtained by analyzing DNA extraction using different types of HI reagents. (d) of FIG. 2 shows the evaluation of CT values for DNA extraction using various amounts of ASAB. (e) of FIG. 2 shows the evaluation of CT values for DNA extraction using various amounts of DMP as the HI reagent. (f) of FIG. 2 shows the examination of CT values for DNA extraction at different incubation temperatures. (g) of FIG. 2 shows the measurement of CT values for DNA extraction at different incubation times. (h) of FIG. 2 shows CT values of DNA extraction using elution buffers with different pH levels.

FIG. 3 shows morphological characteristics of raw and sulfuric acid-treated bentonites under various acid concentrations. (a) of FIG. 3 presents field-emission scanning electron microscopy (FE-SEM) images of raw bentonite and bentonite activated with 1%, 5%, and 10% sulfuric acid. (b) of FIG. 3 presents nitrogen adsorption–desorption isotherms, and (c) of FIG. 3 presents pore size distributions of the same bentonite samples. FIG. 4 shows verification of the surface modification process of bentonite.

FIG. 4 shows Fourier transform infrared (FTIR) spectra; (a) of FIG. 4 corresponds to raw bentonite, and (c) of FIG. 4 corresponds to sulfuric acid-activated bentonite (SAB). Each color represents the initial state (gray), APDMS silanization (red), and amine modification with HI (blue). (b) and (d) of FIG. 4 show zeta potential measurement results, where (b) of FIG. 4 corresponds to raw bentonite and (d) of FIG. 4 corresponds to SAB, with the same color representations as above.

FIG. 5 shows the performance evaluation results of the ASAB-based SPE system for nucleic acid separation. (a) of FIG. 5 shows DNA binding and elution capacity. (b) of FIG. 5 compares DNA purity extracted from E. coli samples (10⁶ CFU/reaction). (c) of FIG. 5 evaluates the versatility of the ASAB-based SPE system for DNA extraction from various organisms. (d) of FIG. 5 shows qPCR results comparing ASAB-based SPE (yellow) and commercial SPE kits (gray) using Brucella ovis samples diluted serially from 10⁶ to 10⁰ CFU. (e) of FIG. 5 shows melting curve analysis of B. ovis amplicons obtained with ASAB-based SPE. (f) of FIG. 5 presents standard curves of qPCR results from ASAB-based (red) and commercial SPE kits (gray). (g) of FIG. 5 shows a schematic workflow of pathogen concentration using the ASAB-based SPE system. (h) of FIG. 5 shows analysis results of the capacity of the ASAB-based SPE system to handle different sample volumes.

FIG. 6 shows the adaptability of the ASAB-based SPE system for DNA extraction from various sample types. (a) and (f) of FIG. 6 show schematic workflows for extracting DNA from urine and plasma, respectively. (b) and (g) of FIG. 6 show qPCR results of DNA extracted from four different organisms using ASAB-based (blue/pink) and commercial SPE kits (gray). (c) and (h) of FIG. 6 show qPCR results of serially diluted B. ovis samples (10⁶–10⁰ CFU) in urine and plasma, respectively. (d) and (i) of FIG. 6 show melting curve analyses of B. ovis amplicons obtained using ASAB-based SPE from urine and plasma, respectively. (e) and (j) of FIG. 6 show standard curves of qPCR results for both ASAB-based (red) and commercial SPE kits (gray).

FIG. 7 shows results of miRNA and viral RNA extraction from cell culture media using ASAB-based SPE. (a) of FIG. 7 shows a schematic workflow for miRNA extraction. (b) of FIG. 7 shows RT-qPCR results for small RNA markers (miR-21-5p, miR-1246, and U6 snRNA) extracted from HCT116 colorectal cancer cell culture media. (c) of FIG. 7 shows melting curve profiles of the three markers from ASAB-based and commercial SPE kits. (d) of FIG. 7 shows a schematic workflow for viral RNA extraction. (e) of FIG. 7 shows RT-qPCR results of RNA extracted from SARS-CoV-2–infected VeroE6 cell media using ASAB-based (red) and commercial (gray) SPE kits. (f) of FIG. 7 shows melting curve profiles of SARS-CoV-2 RNA amplicons obtained by both systems.

FIG. 8 shows the specific surface area and pore size distribution of the modified bentonite according to the present invention. FIG. 9 shows those of modified zeolite (a of FIG. 9), diatomite (b of FIG. 9), montmorillonite (c of FIG. 9), kaolinite (d of FIG. 9), and halloysite (e of FIG. 9).

FIG. 10 shows the real-time PCR CT values of E. coli (10⁵ CFU) DNA samples extracted using different clay minerals (Diatom Earth(DE), Zeolite(Zeo), Bentonite(Bento), Halloysite(Hallo), Montmorillonite(Monto), Kaolinite(Kao)) as solid-phase matrices.

### DETAILED DESCRIPTION OF THE INVENTION

Hereinafter, the present invention will be described in detail.

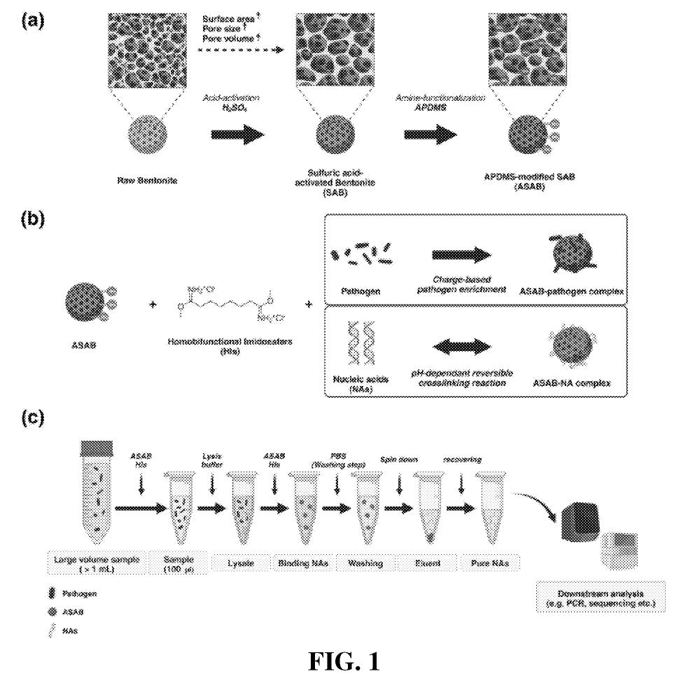

The inventors of the present invention focused on improving the efficiency of nucleic acid extraction by developing a solid-phase extraction (SPE) matrix using sulfuric acid-activated bentonite (SAB). FIG. 1 illustrates the preparation of acid-activated bentonite (ASAB) modified with a silane compound, APDMS, and the nucleic acid extraction process through a pH-dependent reversible crosslinking strategy using the ASAB as a solid phase. Acid activation increases the surface area, pore size, and pore volume of the original bentonite, thereby enhancing the efficiency of amination modification (FIG. 1A). Acid activation not only improves the surface area of bentonite but also promotes surface modification by introducing hydrogen ions into the aluminum and silicon atoms on the bentonite surface, thereby increasing the modification efficiency. Subsequently, the surface of SAB was modified into primary amine groups using APDMS to form ASAB, which was used as the SPE matrix.

Next, homobifunctional imidoesters (HI) are widely used in bio-conjugation applications, such as immobilizing biomolecules onto solid supports for biosensors. The amine groups of ASAB react with HI to form stable amidine bonds, and crosslink with negatively charged nucleic acids or pathogens (FIG. 1B). FIG. 1C illustrates the pathogen concentration and nucleic acid extraction steps of a single-tube sample preparation method. When a large sample volume is mixed with the ASAB–HI complex, negatively charged pathogens are adsorbed onto the complex. The complex is then precipitated by centrifugation, the supernatant is removed, and the sample becomes concentrated into a small volume. Additionally, nucleic acids released from the lysate are crosslinked with the ASAB and HI reagents. HI reacts with the terminal amine groups of nucleic acids to form amidine bonds and crosslinks simultaneously with ASAB, thereby forming an ASAB–HI–nucleic acid complex. This complex remains stable during two washing steps under neutral pH. After washing, the ASAB–HI–nucleic acid complex is decomposed by the elution buffer, providing purified nucleic acid within 30 minutes. The separated nucleic acids can be immediately used for downstream analysis such as real-time PCR (qPCR), and almost no degradation of the nucleic acid occurs due to the high pH throughout the process. Moreover, since the process requires only simple tools such as a pipette and a centrifuge, it offers excellent accessibility and cost efficiency.

To achieve the above purposes, the present invention provides a composition for concentrating nucleic acid or pathogen comprising acid- and silane compound-modified bentonite and homobifunctional imidoesters as active ingredients.

As used herein, the term “acid activation” refers to a process of treating bentonite with a strong acid to remove exchangeable cations and to dissolve a portion of structural components, thereby forming new pores and enlarging existing pores. Through this process, a more uniform pore structure is generated, and the available surface area for adsorption is significantly increased. Bentonite treated with a strong acid exhibits a more uniform surface structure and an increased number of binding-active sites, which greatly enhances its adsorption capacity.

As used herein, the term “bentonite” refers to a type of clay mineral having a large surface area due to its charged surface, hydrophilicity, and microporous volume. Bentonite has been widely studied for removing pollutants such as harmful chemicals, pharmaceuticals, and pesticides. However, its heterogeneous pore size limits reproducible application in separation processes. To address this limitation, the inventors devised a modification method to improve the morphological and chemical characteristics of bentonite through acid activation.

As used herein, the term “silane compound” refers to a compound containing silicon (Si) and hydrogen (H), generally represented by the molecular formula SiHₙ. The simplest form is silane (SiH₄). Silane compounds are chemically highly reactive and easily oxidized in air, and some exhibit spontaneous flammability.

Through silanization of acid-activated bentonite, amino groups can be introduced onto the bentonite surface. This process involves reacting the silanol (Si–OH) groups on the bentonite surface with an amino-containing silane compound, such as 3-aminopropyl(diethoxy)methylsilane (APDMS). During the silanization reaction, the alkoxy groups of the aminosilane are hydrolyzed to form silanol groups, and these newly formed silanol groups undergo condensation reactions with the silanol groups on the bentonite surface. After the reaction, the aminosilane is covalently bonded to the bentonite surface through Si–O–Si linkages, and amino groups (–NH₂) are exposed on the surface. The exposed amino groups impart positive charges to the bentonite. These positive charges allow the bentonite to bind with negatively charged nucleic acids.

As used herein, the term “homobifunctional imidoesters (HI)” refers to reagents that are widely used in bio-conjugation applications such as immobilizing biomolecules onto the solid support of biosensors. The amine groups of ASAB react with HI to form stable amidine bonds, and crosslink with negatively charged nucleic acids or pathogens.

As used herein, the term “nucleic acid” refers to a polymer of deoxyribonucleotides or ribonucleotides having a linear or circular spatial structure, and existing in either a single-stranded or double-stranded form.

In one embodiment of the present invention, the acid may be one or more selected from the group consisting of perchloric acid, hydroiodic acid, hydrobromic acid, hydrochloric acid, sulfuric acid, nitric acid, chloric acid, p-toluenesulfonic acid, methanesulfonic acid, fluoroantimonic acid, fluorosulfonic acid, and triflic acid, and more specifically may be sulfuric acid.

In another embodiment of the present invention, the silane compound may be one or more selected from the group consisting of (3-aminopropyl)triethoxysilane (APTES), (3-aminopropyl)trimethoxysilane, (1-aminomethyl)triethoxysilane, (2-aminoethyl)triethoxysilane, (4-aminobutyl)triethoxysilane, (5-aminopentyl)triethoxysilane, (6-aminohexyl)triethoxysilane, 3-aminopropyl(diethoxy)methylsilane (APDMS), N-[3-(trimethoxysilyl)propyl]ethylenediamine, N-[3-(trimethoxysilyl)propyl]diethylenetriamine, [3-(2-aminoethylamino)propyl]trimethoxysilane (AEAPTMS), and 3-[(trimethoxysilyl)propyl]diethylenetriamine (TMPTA), and more specifically may be 3-aminopropyl(diethoxy)methylsilane (APDMS).

In another embodiment of the present invention, the homobifunctional imidoester may be one or more selected from the group consisting of dimethyl adipimidate (DMA), dimethyl pimelimidate (DMP), dimethyl suberimidate (DMS), and dimethyl 3,3'-dithiobispropionimidate (DTBP), and more specifically may be dimethyl pimelimidate (DMP).

In another embodiment of the present invention, the homobifunctional imidoester may be added at a concentration of 1–20 mg/ml per 1 mL of a biological sample, and more specifically at a concentration of 10 mg/ml.

In another embodiment of the present invention, the nucleic acid may be one or more selected from the group consisting of DNA, RNA, miRNA, mRNA, siRNA, tRNA, sgRNA, and shRNA.

In another embodiment of the present invention, the pathogen may be a microorganism.

In another embodiment of the present invention, the microorganism may be any negatively charged microorganism, and more specifically may be one or more selected from the group consisting of virus, bacterium, fungus, and protozoa. More preferably, the microorganism may be Salmonella enterica (SE), Bacillus cereus (BC), Brucella ovis (BO), or Aspergillus fumigatus (AF) as described in the Examples of the present invention.

The present invention also provides a kit for concentrating nucleic acid or pathogen comprising the above composition.

The kit may further comprise a lysis buffer or protease for lysing cells within a sample to release nucleic acids from inside the cells, and may additionally include buffers for pH adjustment required for effective concentration of pathogens or nucleic acids.

The kit may be in any form selected from the group consisting of a well-plate, tube, column, and microfluidic chip; however, the configuration of the kit is not limited thereto and may include any form commercially available.

The kit may be provided as a single composition or may store individual components of the microorganism-concentrating or nucleic acid-extracting composition separately for mixing prior to use. In this manner, the components of the composition for concentrating pathogens or nucleic acids contained in the kit of the present invention may be packaged separately or as one or more mixtures of arbitrary combinations of components. Each separate component or one or more mixtures thereof may be placed in different containers, or the entire kit may be contained within a single container.

The present invention also provides a method for concentrating nucleic acid or pathogen, comprising: a step of adding and mixing the above composition with a biological sample; and a step of separating nucleic acid or pathogen from the mixture.

In one embodiment of the present invention, the biological sample may be one or more selected from the group consisting of blood, serum, plasma, lymph fluid, cerebrospinal fluid, body fluid, saliva, feces, urine, cerebrospinal fluid, gastric secretion, ascites, nasal secretion, sputum, pharyngeal exudate, and tissue.

As used herein, the term “biological sample” is a broad concept including all kinds of specimens derived from humans or animals. Specifically, the biological sample may include, but is not limited to, blood, serum, plasma, saliva, urine, cerebrospinal fluid, gastric secretion, mucosal samples, peritoneal samples, nasal secretion, sputum, and pharyngeal exudate.

In another embodiment of the present invention, the composition may be added at a concentration of 0.2–5 mg/ml per 1 mL of the biological sample, and more specifically, at a concentration of 1 mg/ml.

In another embodiment of the present invention, the mixing may comprise incubation for 15–60 minutes, and more specifically, for 20 minutes.

In another embodiment of the present invention, the mixing may be performed at 45–60°C, and more specifically, at 56°C.

In another embodiment of the present invention, the separating may comprise injecting a high-pH elution buffer to separate the nucleic acid from the bentonite.

The elution buffer causes the amine groups of the acid-activated bentonite to lose their positive charges, thereby promoting the separation of the pathogen or nucleic acid concentrated on the bentonite surface.

In another embodiment of the present invention, the high-pH elution buffer may have a pH of 10.5–12, and more specifically, a pH of 11.

The present invention also provides a method for preparing bentonite for concentrating nucleic acid or pathogen, comprising: (i) treating bentonite with an acid to obtain activated bentonite; and (ii) modifying the activated bentonite with a silane compound.

In one embodiment of the present invention, the acid may be one or more selected from the group consisting of perchloric acid, hydroiodic acid, hydrobromic acid, hydrochloric acid, sulfuric acid, nitric acid, chloric acid, p-toluenesulfonic acid, methanesulfonic acid, fluoroantimonic acid, fluorosulfonic acid, and triflic acid, and more specifically may be sulfuric acid.

In another embodiment of the present invention, in step (i), the acid treatment may be performed using an acid having a concentration of 0.5–10%, and more specifically, a concentration of 1%.

In another embodiment of the present invention, the silane compound may be one or more selected from the group consisting of (3-aminopropyl)triethoxysilane (APTES), (3-aminopropyl)trimethoxysilane, (1-aminomethyl)triethoxysilane, (2-aminoethyl)triethoxysilane, (4-aminobutyl)triethoxysilane, (5-aminopentyl)triethoxysilane, (6-aminohexyl)triethoxysilane, 3-aminopropyl(diethoxy)methylsilane (APDMS), N-[3-(trimethoxysilyl)propyl]ethylenediamine, N-[3-(trimethoxysilyl)propyl]diethylenetriamine, [3-(2-aminoethylamino)propyl]trimethoxysilane (AEAPTMS), and 3-[(trimethoxysilyl)propyl]diethylenetriamine (TMPTA), and more specifically may be 3-aminopropyl(diethoxy)methylsilane (APDMS).

The present invention also provides a modified bentonite comprising amino (–NH₂) or amine (–NHR) functional groups on the surface.

The amino or amine functional groups may be introduced onto the surface of acid-activated bentonite through a silanization reaction in which a silane compound is reacted with the acid-activated bentonite.

The acid and silane compounds may be the same as those described above.

The modified bentonite may have a specific surface area of, for example, 150 m²/g to 300 m²/g, or 200 m²/g to 250 m²/g. The modified bentonite of the present invention may have an increased surface area, pore size, and pore volume compared with the original bentonite through acid activation treatment.

Specifically, the modified bentonite of the present invention was confirmed to have a higher specific surface area than other modified clay minerals such as montmorillonite or halloysite.

The modified bentonite of the present invention may exhibit a synergistic effect on nucleic acid adsorption due to the high specific surface area and the presence of amino/amine functional groups on the surface.

The present invention will be described in further detail through the following examples. However, these examples are provided for illustrative purposes only and are not intended to limit the scope of the invention in any way.

### EXAMPLE

### 1. Materials and Methods

### 1.1 Materials and Reagents

All oligonucleotides were synthesized by Bionics (Seoul, Korea). The oligonucleotides were dissolved in diethyl pyrocarbonate (DEPC)-treated water to prepare a stock solution with a concentration of 100 μM. Salmon sperm DNA was purchased from Sigma-Aldrich and diluted to various concentrations with DEPC-treated water. Clinically negative pooled human plasma and urine were obtained from Innovative Research (USA). Bentonite, diatomite, zeolite, montmorillonite, kaolinite, and halloysite were purchased from Sigma-Aldrich. Sulfuric acid and anhydrous ethanol were purchased from Daejung Chemicals (Korea). Proteinase K was purchased from Qiagen. Tris-HCl, ethylenediaminetetraacetic acid (EDTA), Triton X-100, sodium dodecyl sulfate (SDS), and sodium hydroxide solution were purchased from Sigma-Aldrich. The cell culture supernatant of VeroE6 cells infected with SARS-CoV-2 was purchased from Zeptometrix.

### 1.2 Acid Activation of Bentonite

Acid-activated bentonite was prepared by mixing 10 g of bentonite with 100 mL of sulfuric acid at 85°C for 4 hours under adequate stirring. The activation process was terminated by adding a large amount of deionized (DI) water. The acid-activated bentonite was washed with DI water until the washing solution reached neutral pH and then dried overnight under vacuum. The dried product was stored at room temperature until further use.

### 1.3 Preparation of Amine-Modified Extraction Matrix

The amine-modified extraction matrix was synthesized by reacting the surface of purified clay minerals with the amine group of silane compounds. Each matrix was purified by a gravity sedimentation method in DI water. To prepare the amine-modified extraction matrix, 3-aminopropyl(diethoxy)methylsilane (APDMS, 97%, Sigma-Aldrich) was used. Five milliliters of the silane were slowly added dropwise to a mixture of 95 mL of anhydrous ethanol under vigorous stirring. Then, 2 g of the purified clay was added. The reaction was maintained at 75°C for 4 hours. The functionalized clay was washed twice with anhydrous ethanol and then dried overnight under vacuum. Finally, the amine-modified dried clay was stored in a desiccator until further analysis.

### 1.4 Characterization

The morphological and chemical characteristics of the amine-modified clay mineral matrix were analyzed using the following instruments: a field-emission scanning electron microscope (FE-SEM, JSM-7001F, JEOL, Tokyo, Japan); a Fourier transform infrared spectrometer (FTIR, VERTEX 70, Bruker Optics Inc., Ettlinger, Germany); a zeta potential analyzer (Nano ZS 90, Malvern Panalytical Ltd., Worcestershire, UK); and a nitrogen adsorption–desorption isotherm analyzer based on the Brunauer–Emmett–Teller (BET) principle (Autosorb-iQ 2ST/MP, Quantachrome, FL, USA).

### 1.5 Cell Culture

Escherichia coli (ATCC 25922), Bacillus cereus (ATCC 10876), and Salmonella enterica (ATCC 14028) were inoculated into nutrient broth medium and cultured overnight at 37°C under shaking conditions. Brucella ovis (ATCC 25840) was cultured on Brucella agar containing 5% defibrinated sheep blood at 37°C under 5% CO₂ for 48 hours. After incubation, bacterial suspensions were quantified using the agar plate method and subsequently diluted to various concentrations with phosphate-buffered saline (PBS). Aspergillus fumigatus (ATCC 36607) was cultured on Sabouraud dextrose agar containing chloramphenicol at 25°C for 5 days. After incubation, A. fumigatus was resuspended in PBS and quantified using a hemocytometer. HCT116 cells were cultured in Dulbecco’s Modified Eagle’s Medium (DMEM, GenDEPOT) supplemented with 10% fetal bovine serum and 1% antibiotic–antimycotic solution at 37°C. The pH of the cell culture medium was maintained by adjusting CO₂ to 5%. After cultivation, the cell suspensions were counted using a hemocytometer and appropriately diluted in 1× PBS.

### 1.6 General Nucleic Acid Extraction Procedure

A schematic procedure for nucleic acid extraction using ASAB is shown in FIG. 1. Dimethyl pimelimidate (DMP, Sigma-Aldrich) was selected as the optimal cross-linking reagent in optimization experiments. For DNA extraction from pathogens, 200 μL of sample was mixed with 20 μL of Proteinase K and 200 μL of a custom-prepared lysis buffer (LB) comprising 100 mM Tris-HCl, 10 mM EDTA, 10% Triton X-100, and 1% SDS adjusted to pH 8.0. Subsequently, 20 μL of ASAB (prepared at a concentration of 50 mg/mL in DI water) and 100 μL of DMP solution (prepared at 100 mg/mL in DI water) were sequentially added. The mixture was incubated at 56°C for 20 minutes with gentle agitation every 5 minutes. After incubation, the sample was centrifuged for 1 minute in a spin-down device, and the supernatant was removed. The precipitate was washed twice with 500 μL of PBS. Finally, 100 μL of 10 mM Tris-HCl (pH > 11, adjusted with NaOH) was added as an elution buffer. After incubation at room temperature for 1 minute, the sample was centrifuged for 1 minute in a spin-down device. The supernatant containing the separated nucleic acid was stored at –20°C until further analysis. The same procedure was applied for DNA extraction from mammalian cells and fungi, as well as for microRNA (miRNA) and viral RNA separation using ASAB. For comparison, the same samples were processed using commercial solid-phase extraction (SPE) kits according to the manufacturer’s instructions (QIAamp DNA Mini Kit, Qiagen). For miRNA and viral RNA extraction, commercially available kits (QIAamp RNA Kit and QIAamp Viral RNA Mini Kit, respectively) were used as standards. The quality and quantity of extracted nucleic acids were determined by measuring the absorbance ratio at 260/280 nm using a spectrophotometer (NanoDrop Lite Plus, Thermo Fisher Scientific), and the cycle threshold (CT) values were evaluated using real-time PCR (CFX96, Bio-Rad).

### 1.7 Bacterial Concentration Process

In the pre-concentration process for pathogens, 40 μL of ASAB (50 mg/mL in DI water) and 200 μL of DMP solution (100 mg/mL in DI water) were sequentially added to 1 mL of sample and mixed. Pathogens were collected by the ASAB–HI complex through gentle agitation at room temperature every 5 minutes for 30 minutes. For large-volume samples (5, 10, 30, and 50 mL), reactions were performed on a rotating mixer (Topscien Instrument Co., Ltd.) at 99 rpm for 30 minutes. After two washes with 1 mL of PBS, the pathogen-attached ASAB–HI complex was collected by centrifugation. The subsequent nucleic acid extraction process was carried out in 1.5 mL tubes.

### 1.8 Real-Time PCR

Real-time PCR (qPCR) was performed to determine the quality of the separated nucleic acids. For DNA amplification, 5 μL of the extracted DNA was amplified in a total reaction volume of 20 μL containing 2× AccuPower GreenStar™ qPCR PreMix (Bioneer), 500 nM primers, and DI water. The PCR cycling conditions were as follows: an initial denaturation at 95°C for 10 minutes; followed by 40 cycles of 95°C for 15 seconds and 60°C for 30 seconds for annealing and extension. For RNA amplification, 5 μL of the extracted RNA was amplified in a total reaction volume of 20 μL containing 2× AccuPower® GreenStar™ RT-qPCR Master Mix (Bioneer), 500 nM of each primer, and DI water. The PCR cycling conditions were as follows: reverse transcription at 50°C for 20 minutes; initial denaturation at 95°C for 10 minutes; followed by 40 cycles of 95°C for 30 seconds and 60°C for 30 seconds. Prior to microRNA amplification, complementary DNA (cDNA) was synthesized using the Mir-X™ miRNA First-Strand Synthesis Kit (Takara Bio) according to the manufacturer’s protocol. Briefly, 3.75 μL of the extracted RNA was mixed in a 10 μL reaction mixture containing 1.25 μL of mRQ enzyme and 5 μL of 2× mRQ buffer. The thermal conditions for cDNA synthesis were as follows: cDNA synthesis at 37°C for 60 minutes and termination for enzyme inactivation at 85°C for 5 minutes. Then, 90 μL of DI water was added to the synthesized cDNA sample, which was stored at –80°C for later use. For cDNA amplification, the MiR-X™ miRNA qRT-PCR TB Green® Kit (Takara Bio) was used according to the manufacturer’s protocol. For U6 snRNA amplification, the PCR cycling conditions were as follows: initial denaturation at 95°C for 10 seconds, followed by 40 cycles of denaturation at 95°C for 5 seconds and annealing/extension at 60°C for 20 seconds. All amplified products underwent melting curve analysis at intervals of 0.5°C from 65°C to 95°C for 5 seconds each. The fluorescence signals of the amplified products were obtained using a CFX96 real-time PCR system.

### 2. Experimental Results

### 2.1 Optimization of the Acid-Activated Bentonite-Based Solid-Phase Extraction (SPE) System for Nucleic Acid Extraction

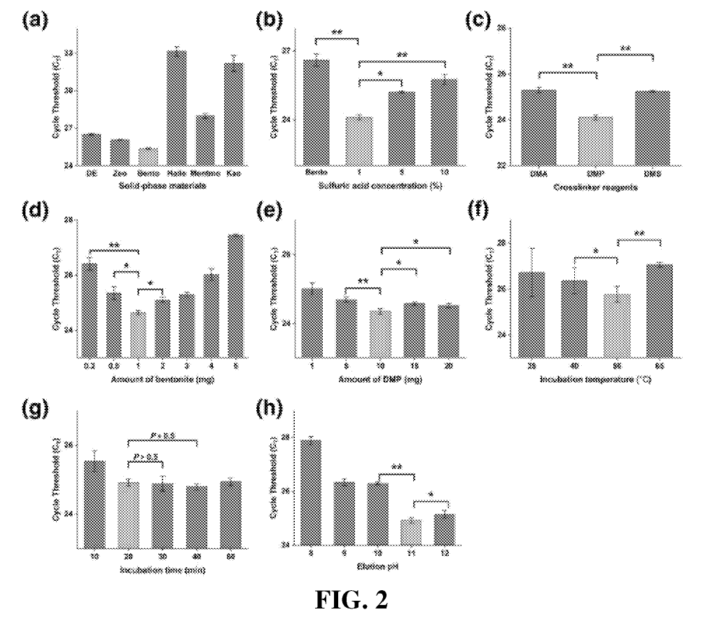

The inventors optimized the solid-phase extraction (SPE) system of the present invention by adjusting all components and procedures (FIG. 2). Nucleic acids were separated using ASAB and HI, and the extracted nucleic acids were quantified by quantitative PCR (qPCR). Under constant experimental conditions, nucleic acids were extracted from E. coli, and factors yielding the lowest cycle threshold (CT) values, corresponding to the highest nucleic acid recovery, were identified by varying several parameters.

The inventors compared various clay minerals—diatomite, zeolite, bentonite, montmorillonite, kaolinite, and halloysite—as potential solid-phase matrices for nucleic acid extraction (FIG. 2A). According to qPCR results, bentonite showed 1.6- to 221-fold higher nucleic acid recovery compared with other clay minerals. Subsequently, the bentonite was modified with sulfuric acid at three concentrations (1%, 5%, and 10%) to enhance its adsorption capacity, and the 1% acid activation was found to be the most effective condition for use as an SPE matrix (FIG. 2B).

In this study, the inventors employed a pH-dependent crosslinking strategy using three types of HI reagents—dimethyl suberimidate (DMS), dimethyl adipimidate (DMA), and dimethyl pimelimidate (DMP)—and evaluated the DNA recovery efficiency based on qPCR results. Among these, DMP exhibited an average of 2.2-fold higher nucleic acid recovery than the other HI reagents and was therefore selected as the crosslinking reagent for the SPE system of the present invention (FIG. 2C). The superior DNA separation efficiency of DMP is presumed to be due to its optimal carbon chain length (nine carbons) that provides a balanced combination of flexibility and rigidity (compared to DMA: seven carbons, and DMS: eleven carbons), which promotes efficient pH-dependent reversible crosslinking.

Excessively increasing the surface area of the reaction mixture may induce nonspecific adsorption, thereby affecting nucleic acid separation efficiency. As a result of optimizing the amount of ASAB, 1 mg of ASAB provided optimal performance based on the qPCR results (FIG. 2D). The inventors closely examined the factors influencing the imidoester–primary amine reaction, including pH, temperature, and reagent concentration, and optimized the amount of DMP, incubation temperature and time for DNA binding, and pH of the elution buffer. As a result, 10 mg of DMP per reaction showed the best performance in DNA separation (FIG. 2E).

The optimal incubation temperature was determined to be 56°C, based on delayed CT values observed at 40°C and 65°C, which indicated low lysis buffer activity and inhibition of the crosslinking reaction, respectively (FIG. 2F). Extending the incubation time beyond 20 minutes produced no significant difference; therefore, 20 minutes was selected as the optimal incubation time (FIG. 2G).

Finally, the inventors examined the elution buffer optimized for reversing the crosslinking reaction, which is critical for releasing the separated DNA. The crosslinking reaction induced by HI was stable at neutral pH but reversible at high pH (FIG. 2H). Considering the instability of DNA double helices at excessively high pH, the inventors determined that an elution buffer at pH 11 provided the highest extraction efficiency.

In summary, the inventors maximized nucleic acid recovery through the combination of bentonite and DMP and effectively optimized experimental parameters such as DMP concentration and incubation conditions. The importance of pH adjustment in the elution buffer was confirmed, showing that high-pH conditions enable reversible crosslinking and improve DNA extraction efficiency. These results demonstrate that the solid-phase extraction system of the present invention exhibits excellent performance in nucleic acid separation and provides potential applicability in a variety of analytical fields.

### 2.2 Development of Solid-Phase Extraction Matrix through Morphological Study of Acid-Activated Bentonite

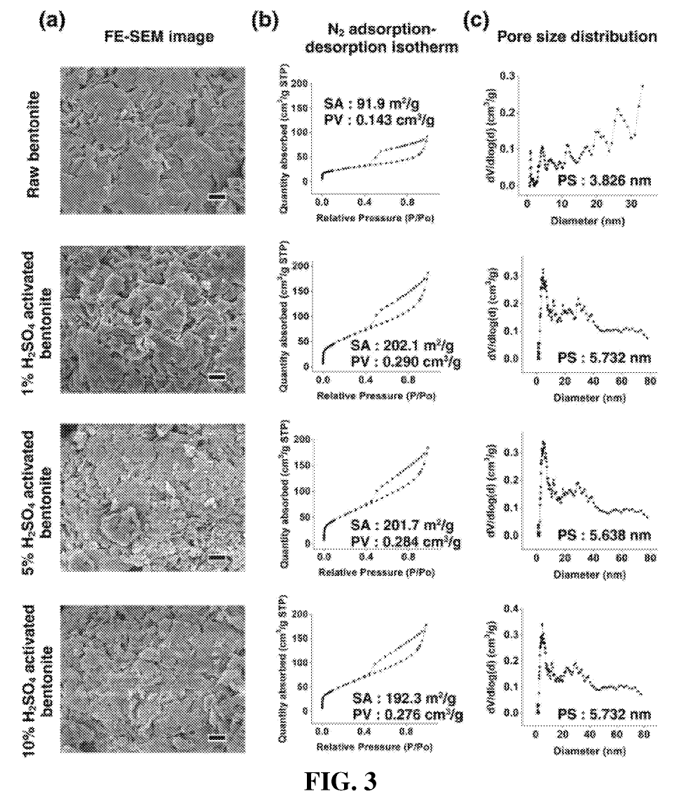

A high surface area is one of the essential requirements for an SPE matrix. To better understand the improved extraction efficiency associated with the acid modification process of the bentonite surface, the inventors investigated the morphological characteristics of bentonite before and after amine modification at various sulfuric acid concentrations (1%, 5%, and 10%) (FIG. 3). A field-emission scanning electron microscope (FE-SEM) was used to study the particle size and morphology, and the textural properties (surface area, pore volume, and pore size distribution) were analyzed by nitrogen adsorption–desorption isotherms based on the Brunauer–Emmett–Teller (BET) principle. FE-SEM images for all bentonite conditions were obtained at the same magnification (×10,000). The FE-SEM images indicated that all raw bentonite samples exhibited a coarse surface morphology characteristic of the clay mineral group.

No significant morphological differences were observed among the acid-treated bentonite samples (FIG. 3A). However, after amine modification with APDMS, FE-SEM images for all conditions showed smoother surface textures, suggesting successful chemical modification. These microscopic observations indicate that the bentonite modified with APDMS was successfully surface-functionalized for biological applications. According to the nitrogen (N₂) adsorption–desorption isotherm results, raw bentonite had a surface area (SA) of 91.9 m²/g, a pore volume (PV) of 0.143 cm³/g, and a pore size (PS) of 3.826 nm (FIGS. 3B and 3C). Bentonite modified with three sulfuric acid concentrations (1%, 5%, and 10%) exhibited SA/PV/PS values of 202.1/0.290/5.732, 201.7/0.284/5.638, and 192.3/0.276/5.732 m²/g·cm³/g·nm, respectively, showing that the surface area increased approximately 2.2-fold compared with raw bentonite.

For comparison, diatomite and zeolite previously used as SPE materials exhibited SA/PV/PS values of 15.70/0.019/4.752 and 13.32/0.018/5.425 m²/g·cm³/g·nm, respectively. These results indicate that the average surface area of sulfuric acid-activated bentonite (SAB) was approximately 12.66 times greater than that of diatomite and 14.92 times greater than that of zeolite. This increased surface area enhances the efficiency of chemical functionalization and adsorption capacity, explaining the high nucleic acid extraction efficiency observed when SAB is used as the SPE matrix.

In summary, the improved surface morphology confirmed by FE-SEM imaging and the significant enhancement of surface area and pore structure confirmed by nitrogen adsorption–desorption isotherm analysis demonstrate that sulfuric acid treatment effectively improves the textural properties of bentonite. The increase in surface area according to the acid concentration enhances the adsorption capacity and the efficiency of chemical functionalization. The inventors revealed that bentonite possesses a remarkably higher surface area than diatomite and zeolite, which correlates strongly with the high nucleic acid recovery rate observed in solid-phase extraction. These findings expand the potential use of bentonite as an SPE matrix and contribute to establishing an efficient nucleic acid extraction system for various biological applications.

### 2.3 Enhancement of Nucleic Acid Binding Affinity through Amine Modification and Acid Activation of Bentonite

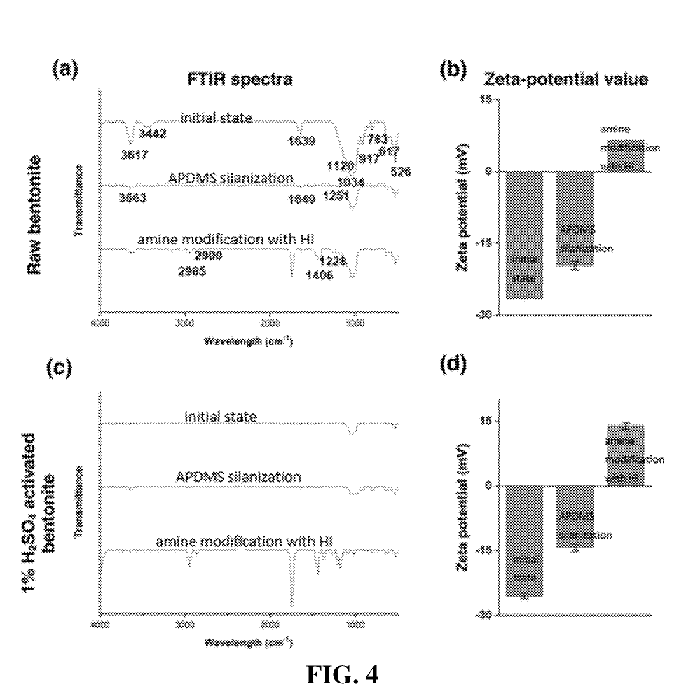

Another important requirement for solid-phase extraction (SPE) technology is the surface functionalization process. The inventors confirmed that the amine modification of sulfuric acid-activated bentonite (SAB) was successfully achieved through Fourier-transform infrared (FTIR) spectroscopy and zeta potential (mV) measurements (FIG. 4). Acid activation modifies the surface of the material without altering the original composition, which was evidenced by the unchanged FTIR spectra between raw bentonite and SAB. Both raw bentonite and SAB exhibited absorption peaks at 526, 783, and 1120 cm⁻¹ corresponding to the asymmetric stretching vibrations of Si–O–Si. The presence of amorphous SiO₂ was indicated by the bands at 1034 and 1634 cm⁻¹. The peaks at 617 and 675 cm⁻¹ were attributed to aluminum–oxygen–silicon–oxygen bonds, suggesting the presence of feldspar. Additionally, the peak at 917 cm⁻¹ represented Al–Al–OH bonding, while the absorption peaks at 1634, 3442, and 3617 cm⁻¹ were attributed to water molecules. After modification with APDMS, new absorption peaks appeared at 1228, 1251, 1649, and 3663 cm⁻¹, indicating the successful introduction of amine groups onto the bentonite surface. The peaks at 1649 and 3663 cm⁻¹ corresponded to N–H stretching vibrations, and those at 1228 and 1251 cm⁻¹ corresponded to C–N stretching vibrations of the amine groups directly bound to the bentonite. In the presence of HI reagents, peaks observed at 2900 and 2985 cm⁻¹ were assigned to C–H stretching vibrations, the peak at 1228 cm⁻¹ to C–N stretching, and the peak at 1406 cm⁻¹ to C–O stretching, all of which were attributed to amidine groups crosslinked to the APDMS-modified bentonite (FIGS. 4A and 4C).

Next, the inventors analyzed the zeta potentials of raw bentonite and SAB samples at each surface modification step to investigate the effect of acid activation on surface functionalization (FIGS. 4B and 4D). The average zeta potentials of raw bentonite and SAB were measured as –26.5 ± 0.36 mV and –25.67 ± 0.57 mV, respectively, indicating that acid activation did not alter the chemical composition of bentonite (gray bars in FIGS. 4B and 4D). The main effect of acid activation was to enhance the physical characteristics, such as surface area, for improved chemical modification rather than altering chemical composition. APDMS-modified raw bentonite and ASAB showed zeta potentials of –19.63 ± 0.86 mV and –14.27 ± 0.87 mV, respectively (red bars in FIGS. 4B and 4D). Compared with APDMS-modified raw bentonite, the higher positive shift observed in ASAB indicated that the acid activation increased surface area, providing more sites for modification and improving the efficiency of amine functionalization. In the subsequent modification step, the APDMS-modified bentonite was reacted with HI reagents to form covalent bonds between the amine groups introduced by APDMS and the imidoester molecules. This step plays an important role in enhancing the binding affinity toward nucleic acids that possess negatively charged phosphate groups. The zeta potential of the HI-bound, APDMS-modified raw bentonite shifted to +6.52 ± 0.24 mV, while that of HI-bound ASAB increased further to +13.9 ± 0.79 mV (blue bars in FIGS. 4B and 4D). This indicates that, due to the increased surface area and pore volume provided by sulfuric acid activation, ASAB exhibits superior surface modification capability. This significant positive shift demonstrates a successful coupling process in which HI molecules neutralize remaining negative charges and introduce net positive charges on the surface, forming a highly functionalized surface with strong nucleic acid binding properties.

In summary, the inventors confirmed the successful introduction of amine groups onto the bentonite surface through FTIR spectral analysis, which clearly showed the appearance of new absorption peaks. Zeta potential measurements demonstrated that acid activation did not alter the chemical composition of bentonite but increased surface area, thereby improving the efficiency of amine modification. The positive shift in zeta potential during the HI coupling process indicated that the modified bentonite enhanced its binding affinity toward nucleic acids. These results confirmed that the SPE system of the present invention successfully implemented a functional surface enabling high-efficiency nucleic acid extraction and is expected to provide an effective approach for future biological applications.

### 2.4 Improvement in Nucleic Acid Extraction Efficiency and Large-Sample Processing Capability of the Acid-Activated Bentonite-Based SPE System

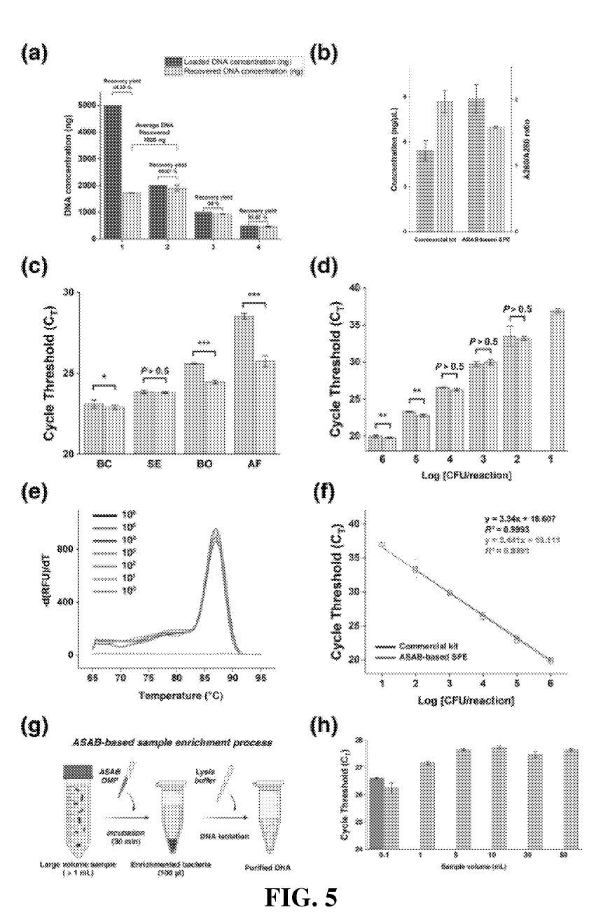

The performance of the nucleic acid extraction platform was evaluated based on yield, purity, and scalability (FIG. 5). To evaluate the binding capacity of the ASAB-based SPE system, purified salmon sperm DNA samples (500, 1000, 2000, and 5000 ng) were added to the system and recovered using the elution buffer (FIG. 5A). The recovery yield was determined by calculating the ratio of total recovered DNA to total loaded DNA, taking into account both adsorption and desorption on the ASAB surface. The binding capacity of the system was confirmed when the recovered DNA reached saturation with increasing loaded DNA (average 1820 ng). The recovery yield of the ASAB-based SPE system was 94.11% (FIG. 5A), which was approximately 1.5 times higher than the yield (61 ± 11%) previously reported for commercial SPE kits utilizing silica surfaces. The absorbance ratio (260/280) obtained from the ASAB-based SPE system was similar to that obtained using a commercial SPE kit (QIAamp DNA Mini Kit) (FIG. 5B). In addition to comparable purity, the ASAB-based SPE system exhibited a 1.63-fold higher absorbance spectrum, indicating a higher DNA yield compared to the commercial kit (FIG. 5B).

Next, the inventors evaluated the nucleic acid extraction capability of the ASAB-based SPE system for various bacteria. DNA was extracted from four different microorganisms using both the ASAB-based SPE system and a commercial SPE kit (FIG. 5C). According to qPCR results, the ASAB-based SPE system extracted 2.05 times more bacterial DNA from 10⁵ CFU bacteria than the commercial SPE kit. Melting curve analysis confirmed a single amplification product in both platforms. Subsequently, the inventors evaluated the ASAB-based SPE system for DNA extraction from bacterial samples ranging from 10⁰ to 10⁶ CFU per reaction using Brucella ovis (FIG. 5D). The results indicated that the detection limit of the ASAB-based SPE system was 10¹ CFU per reaction, which was ten times more sensitive than that of the commercial DNA extraction kit. Melting curve profiling confirmed the correct target amplification for both platforms (FIG. 5E), and the mean distance between standard curves was 0.496, indicating that the ASAB-based SPE system extracted 1.4 times more DNA than the commercial kit (FIG. 5F).

The inventors also evaluated the capacity of the system to process large sample volumes to improve the detection limit for diagnostic applications. Clinically relevant samples are typically collected in volumes ranging from several milliliters to tens of milliliters depending on the source (e.g., blood or urine), but only small portions are generally processed due to limited handling capability. Enhancing the ability to process large volumes increases sensitivity by enabling the capture of low-concentration pathogens. To this end, the inventors integrated a pre-incubation step for sample concentration into the proposed SPE system (FIG. 5G). Initially, ASAB and DMP were added to large-volume samples and incubated at room temperature for 30 minutes. The positively charged ASAB–DMP complexes electrostatically attracted the negatively charged bacterial membranes due to their low isoelectric points. After binding, the precipitated ASAB–DMP complexes were separated by centrifugation, and the subsequent nucleic acid extraction was performed using the ASAB-based SPE system. The inventors processed bacterial DNA from samples with the same CFU concentration (10⁴ CFU per reaction) across various sample volumes (0.1, 1, 5, 10, 30, and 50 mL). According to qPCR results, the concentration approach of the present invention enabled 48% recovery efficiency from a 50 mL sample compared to the total bacterial DNA extracted from a 0.1 mL sample using a commercial SPE kit (FIG. 5H). This result highlights that the ASAB-based SPE system can handle substantially larger sample volumes than commercial SPE kits, which are typically limited to several hundred microliters.

In summary, the ASAB-based SPE system provided a high DNA recovery yield (94.11%) and purity, showing 1.5-fold higher efficiency and 10-fold greater sensitivity compared with commercial SPE kits. According to qPCR analysis, the system extracted 2.05 times more DNA from 10⁵ CFU bacteria and achieved 48% recovery efficiency from 50 mL samples, demonstrating its scalability and superior large-volume handling capability. These findings confirm that the nucleic acid extraction system of the present invention is an effective tool combining high yield and large-scale processing capability suitable for a wide range of applications.

### 2.5 High Extraction Efficiency in Biological Fluids and Enhanced microRNA Extraction Capability of the Acid-Activated Bentonite-Based SPE System

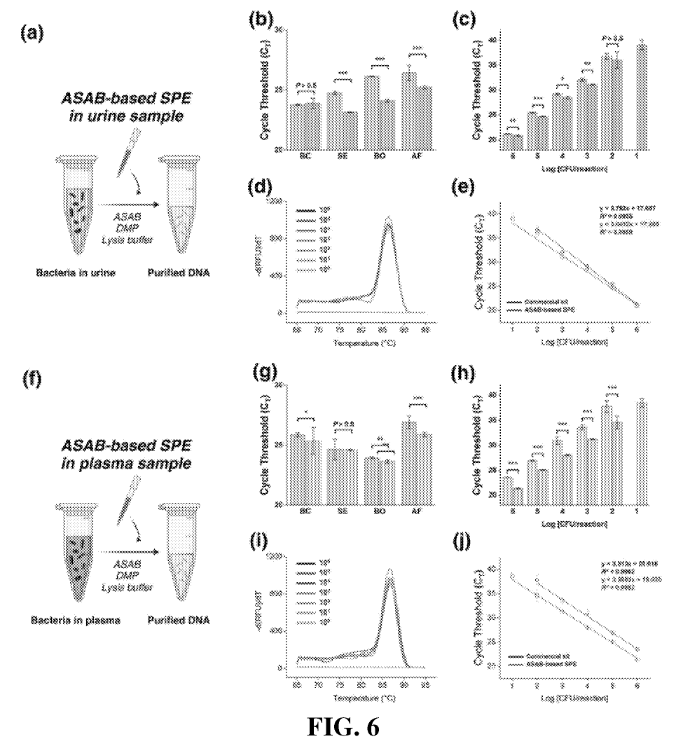

Although the ASAB-based SPE system of the present invention exhibited excellent performance for bacterial DNA extraction in saline buffer, its nucleic acid extraction efficiency in biological fluids can be affected by various factors such as bacterial load, physiological conditions, and the presence of proteins. The bacterial, fungal, or viral load varies depending on the severity and duration of infection and the immune status of the host (typically ranging from 10³ to 10⁷ CFU/mL). Considering these aspects, the inventors evaluated the efficiency of the ASAB-based SPE system for nucleic acid extraction from biological samples. The system was tested in pooled human urine and plasma samples (Innovative Research, USA) using four representative microorganisms: Salmonella enterica (SE), Bacillus cereus (BC), Brucella ovis (BO), and Aspergillus fumigatus (AF) (FIG. 6).

### (1) Evaluation of Nucleic Acid Extraction Efficiency in Biological Fluids

Under the optimized conditions of the ASAB-based SPE system, the inventors extracted bacterial and fungal DNA from pooled human urine and plasma samples (FIGS. 6A and 6F). The qPCR results revealed no significant difference in CT values between urine and PBS-spiked samples, whereas samples spiked into plasma showed an average delay of 0.63 in CT values. Nevertheless, the DNA extracted using the ASAB-based SPE system exhibited faster CT values than those obtained with a commercial SPE kit—by an average of 1.18 cycles in urine and 0.47 cycles in plasma (FIGS. 6B and 6G). Next, Brucella ovis samples serially diluted from 10⁶ to 10⁰ CFU per reaction were processed using the ASAB-based SPE system in human urine and plasma (FIGS. 6C and 6H). The bacterial load of Brucella infection is generally within the range of 10³ to 10⁹ CFU/mL in human urine or plasma. The inventors successfully detected 10¹ CFU per reaction using qPCR, demonstrating ten times higher extraction performance than the commercial SPE kit. Melting curve analysis showed no nonspecific fluorescence signals, confirming clinical negativity in biological fluid samples (FIGS. 6D and 6I). The relationship between the CT values of DNA extracted using the ASAB-based SPE system and pathogen concentration exhibited a high linear correlation (R² = 0.9959 in human urine and R² = 0.9982 in human plasma) (FIGS. 6E and 6J). In particular, in human plasma, the mean distance between standard curves was 1.981, indicating that the ASAB-based SPE system provided approximately 3.95-fold higher DNA yield compared with the commercial SPE kit (FIG. 6J). These results emphasize the superiority of the ASAB-based SPE system in efficiently separating nucleic acids from biological fluids compared with commercial systems.

### (2) Evaluation of microRNA Extraction Efficiency

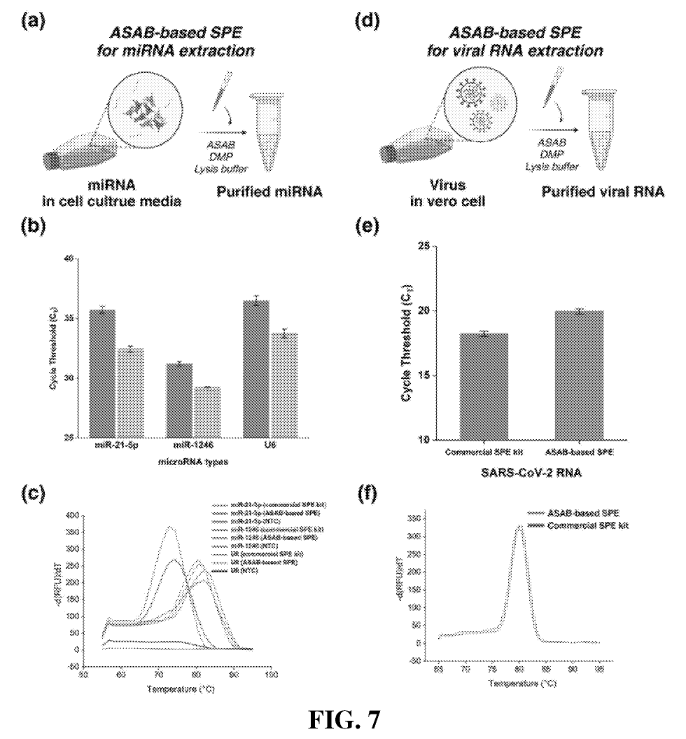

Many practitioners using nucleic acid extraction technologies require solutions for extracting not only DNA but also RNA, including microRNAs (miRNAs). Unlike DNA, RNA is single-stranded and structurally unstable, making it difficult to extract efficiently. The inventors evaluated the potential of the ASAB-based SPE system for extracting microRNAs, which are important regulators associated with disease progression. The analysis of microRNA expression patterns is essential in molecular biology studies and requires high-quality separation technologies. Using the ASAB-based SPE system, microRNAs were extracted from the culture supernatant of HCT116 colorectal cancer cells for the small RNA markers miR-21-5p, miR-1246, and U6 snRNA (FIG. 7A). These markers are known to be highly expressed in tumors. The ASAB-based SPE system exhibited an average of 6.3-fold higher microRNA recovery compared with the commercial RNA extraction kit, including 9.8-fold for miR-21-5p, 3.9-fold for miR-1246, and 6.6-fold for U6 snRNA (FIG. 7B). Furthermore, both the commercial SPE kit and the ASAB-based SPE system displayed similar melting curve profiles for the three markers (FIG. 7C). In addition, to evaluate the broad applicability of the ASAB-based SPE system, RNA extraction efficiency was compared with that of a viral RNA-specific commercial kit (QIAamp Viral RNA Mini Kit) using reverse transcription quantitative PCR (RT-qPCR) (FIG. 7D). The results showed similar melting curve profiles, although the viral RNA-specific kit exhibited CT values 1.7 cycles faster than the ASAB-based system (FIGS. 7E and 7F). These findings indicate that the ASAB-based SPE system is a versatile platform applicable not only to bacterial DNA extraction but also to the separation of unstable and low-abundance small RNA fragments.

In conclusion, the ASAB-based SPE system developed in this study demonstrated high nucleic acid extraction efficiency not only in saline buffer but also in biological fluids, effectively separating bacterial and fungal DNA from various microorganisms. Compared with commercial SPE kits, the system achieved approximately 3.95-fold higher DNA yield and exhibited tenfold higher sensitivity by detecting Brucella infection at 10¹ CFU per reaction. Furthermore, in microRNA extraction, the ASAB-based SPE system exhibited an average of 6.3-fold higher recovery rate and was also applicable to RNA extraction, including viral RNA. These results highlight that the ASAB-based SPE system provides an efficient and universal solution for nucleic acid separation from diverse biological samples.

### 2.6 Comparison of Specific Surface Area between the Modified Bentonite and Other Clay Minerals

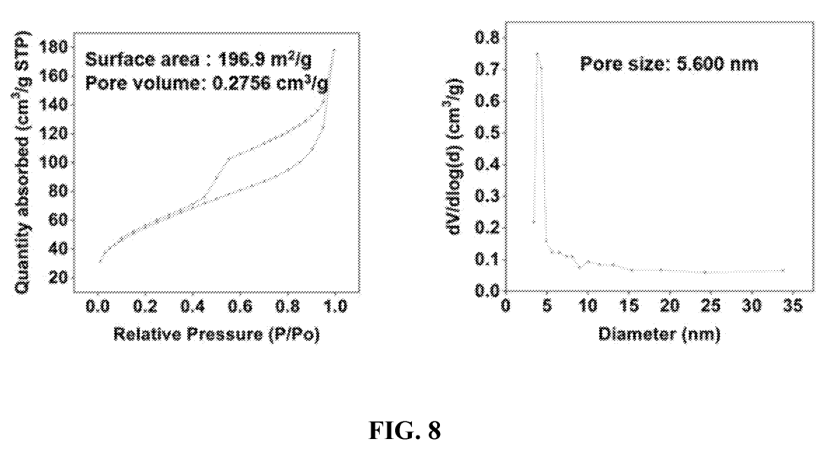

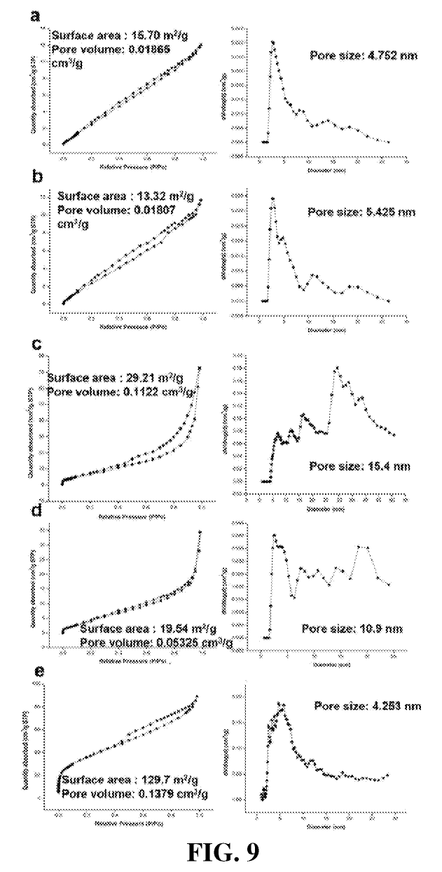

The modified bentonite (acid-activated bentonite) of the present invention exhibited a high specific surface area of approximately 200 m²/g (FIG. 8).

For comparison, the specific surface area and pore size of similarly modified clay minerals—zeolite, diatomite, montmorillonite, kaolinite, and halloysite—were also analyzed (FIG. 9).

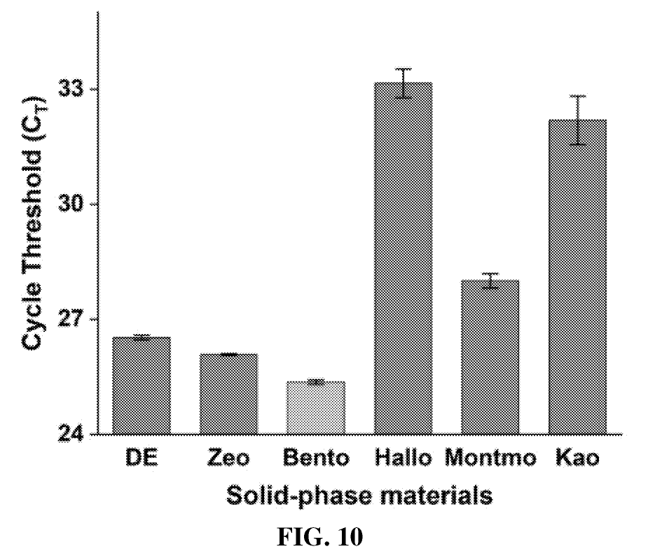

Furthermore, each acid-activated clay mineral was modified with APDMS and crosslinked with homobifunctional imidoesters. Afterward, nucleic acids were attached to the materials under neutral pH conditions and eluted under high-pH conditions. The DNA eluted from each clay mineral was analyzed by real-time PCR, and the CT values were compared (FIG. 10). As shown in FIG. 10, among the various clay minerals tested, the use of bentonite as a solid-phase material resulted in the recovery of the highest amount of DNA, as indicated by the lowest CT value. From these results, it was confirmed that the specific surface area is a key factor in solid-phase extraction performance.

What is claimed is:

1. A composition for concentrating nucleic acid or pathogen, comprising acid- and silane compound-modified bentonite and homobifunctional imidoesters as active ingredients.

2. The composition according to claim 1,
wherein the acid is one or more selected from the group consisting of perchloric acid, hydroiodic acid, hydrobromic acid, hydrochloric acid, sulfuric acid, nitric acid, chloric acid, p-toluenesulfonic acid, methanesulfonic acid, fluoroantimonic acid, fluorosulfonic acid, and triflic acid.

3. The composition according to claim 1,
wherein the silane compound is one or more selected from the group consisting of (3-aminopropyl)triethoxysilane (APTES), (3-aminopropyl)trimethoxysilane, (1-aminomethyl)triethoxysilane, (2-aminoethyl)triethoxysilane, (4-aminobutyl)triethoxysilane, (5-aminopentyl)triethoxysilane, (6-aminohexyl)triethoxysilane, 3-aminopropyl(diethoxy)methylsilane (APDMS), N-[3-(trimethoxysilyl)propyl]ethylenediamine, N-[3-(trimethoxysilyl)propyl]diethylenetriamine, [3-(2-aminoethylamino)propyl]trimethoxysilane (AEAPTMS), and 3-[(trimethoxysilyl)propyl]diethylenetriamine (TMPTA).

4. The composition according to claim 1,
wherein the homobifunctional imidoester is one or more selected from the group consisting of dimethyl adipimidate (DMA), dimethyl pimelimidate (DMP), dimethyl suberimidate (DMS), and dimethyl 3,3'-dithiobispropionimidate (DTBP).

5. The composition according to claim 1,
wherein the nucleic acid is one or more selected from the group consisting of DNA, RNA, miRNA, mRNA, siRNA, tRNA, sgRNA, and shRNA.

6. The composition according to claim 1,
wherein the pathogen is a microorganism.

7. The composition according to claim 6,
wherein the microorganism is one or more selected from the group consisting of virus, bacterium, fungus, and protozoa.

8. A kit for concentrating nucleic acid or pathogen,
comprising the composition according to claim 1.

9. A method for concentrating nucleic acid or pathogen,
comprising adding and mixing the composition according to claim 1 with a biological sample, and separating nucleic acid or pathogen from the mixture.

10. The method according to claim 9,
wherein the biological sample is one or more selected from the group consisting of blood, serum, plasma, lymph fluid, cerebrospinal fluid, body fluid, saliva, feces, urine, cerebrospinal fluid, gastric secretion, ascites, nasal secretion, sputum, pharyngeal exudate, and tissue.

11. The method according to claim 9,
wherein the composition is added at a concentration of 0.2–5 mg/ml per 1 mL of the biological sample.

12. The method according to claim 9,
wherein the mixing comprises incubation for 15–60 minutes.

13. The method according to claim 9,
wherein the mixing is performed at 45–60°C.

14. The method according to claim 9,
wherein the separating comprises injecting a high-pH elution buffer to separate nucleic acid from the bentonite.

15. The method according to claim 14,
wherein the high-pH elution buffer has a pH of 10.5–12.

16. A method for preparing bentonite for concentrating nucleic acid or pathogen, comprising:
(i) treating bentonite with an acid to obtain activated bentonite; and
(ii) modifying the activated bentonite with a silane compound.

17. The method according to claim 16,
wherein the acid is one or more selected from the group consisting of perchloric acid, hydroiodic acid, hydrobromic acid, hydrochloric acid, sulfuric acid, nitric acid, chloric acid, p-toluenesulfonic acid, methanesulfonic acid, fluoroantimonic acid, fluorosulfonic acid, and triflic acid.

18. The method according to claim 16,
wherein the acid in step (i) is used at a concentration of 0.5–10%.

19. The method according to claim 16,
wherein the silane compound is one or more selected from the group consisting of (3-aminopropyl)triethoxysilane (APTES), (3-aminopropyl)trimethoxysilane, (1-aminomethyl)triethoxysilane, (2-aminoethyl)triethoxysilane, (4-aminobutyl)triethoxysilane, (5-aminopentyl)triethoxysilane, (6-aminohexyl)triethoxysilane, 3-aminopropyl(diethoxy)methylsilane (APDMS), N-[3-(trimethoxysilyl)propyl]ethylenediamine, N-[3-(trimethoxysilyl)propyl]diethylenetriamine, [3-(2-aminoethylamino)propyl]trimethoxysilane (AEAPTMS), and 3-[(trimethoxysilyl)propyl]diethylenetriamine (TMPTA).

20. A modified bentonite comprising an amino (-NH₂) or amine (-NHR) functional group on the surface.

21. The modified bentonite according to claim 20,
wherein the modified bentonite has a specific surface area of 150 m²/g to 300 m²/g.
